(19) United States
(12) Patent Application Publication
(10) Pub. No.: US 2025/0290882 A1
(43) Pub. Date: Sep. 18, 2025

(54) THERMAL CONDUCTIVITY GAS SENSOR SYSTEM AND SENSING METHOD

(71) Applicant: KABUSHIKI KAISHA TOSHIBA, Tokyo (JP)

(72) Inventors: Hiroshi HAMASAKI, Hiratsuka Kanagawa (JP); Yui YAMAZAKI, Ota Tokyo (JP); Yosuke AKIMOTO, Yokohama Kanagawa (JP)

(73) Assignee: KABUSHIKI KAISHA TOSHIBA, Tokyo (JP)

(21) Appl. No.: 19/040,937

(22) Filed: Jan. 30, 2025

(30) Foreign Application Priority Data
Mar. 13, 2024 (JP) 2024-038592

(51) Int. Cl. G01N 25/18; G01N 33/00

(52) U.S. Cl. CPC G01N 25/18; G01N 33/0027

(57) ABSTRACT

A thermal conductivity gas sensor system according to an embodiment includes: a measurement unit that includes a sensor unit capable of measuring a resistance value and a heater unit capable of heating the sensor unit; a control unit that includes a heater control circuit capable of controlling heating and non-heating of the heater unit and a timing control circuit capable of controlling timing of a control signal for heating and non-heating of the heater unit and timing of an output signal from the sensor unit; and a calculation unit that is configured to acquire a first output signal from the sensor unit when the heater unit is not heated and correct a second output signal from the sensor unit when the heater unit is heated using the first output signal.

### CROSS-REFERENCE TO RELATED APPLICATION

This application is based upon and claims the benefit of priority from Japanese Patent Application No. 2024-038592, filed Mar. 13, 2024, the entire contents of which are incorporated herein by reference.

### FIELD

Embodiments of the present invention relate generally to a thermal conductivity gas sensor system and a sensing method.

### BACKGROUND

As a gas sensor, there is known a thermal conductivity gas sensor that detects a gas concentration by using two sensors including a detection sensor that detects a change in thermal conductivity while being heated by a heater and an environmental temperature sensor that corrects the gas detection sensor with reference to an environmental temperature.

In such a conventional thermal conductivity gas sensor, since the temperature of the environmental temperature sensor and the temperature of the detection sensor do not match, residuals other than those depending on the gas concentration remain after temperature correction, leading to a decrease in accuracy and sensitivity.

### DESCRIPTION OF THE DRAWINGS

FIG. 1 is a block diagram illustrating a configuration of a thermal conductivity gas sensor system according to a first embodiment.

FIG. 2 is a flowchart related to a sensing method according to the first embodiment.

FIG. 3 is a timing chart related to a sensing method according to the first embodiment.

FIG. 4 is a block diagram illustrating a configuration of a thermal conductivity gas sensor system according to a modification of the first embodiment.

FIG. 5 is a flowchart related to a sensing method according to the modification of the first embodiment.

FIG. 6A is a sectional view illustrating the configuration of the thermal conductivity gas sensor system according to Example 1.

FIG. 6B is a circuit diagram illustrating the configuration of the thermal conductivity gas sensor system according to Example 1.

FIG. 7 is a result of sensing according to Example 1.

FIG. 8 is a result of sensing according to Example 2.

FIG. 9 is a result of sensing according to Comparative Example 1.

### DETAILED DESCRIPTION

A thermal conductivity gas sensor system according to an embodiment includes: a measurement unit that includes a sensor unit capable of measuring a resistance value and a heater unit capable of heating the sensor unit; a control unit that includes a heater control circuit capable of controlling heating and non-heating of the heater unit and a timing control circuit capable of controlling timing of a control signal for heating and non-heating of the heater unit and timing of an output signal from the sensor unit; and a calculation unit that is configured to acquire a first output signal from the sensor unit when the heater unit is not heated and correct a second output signal from the sensor unit when the heater unit is heated using the first output signal.

Hereinafter, a first embodiment will be described with reference to the drawings. The first embodiment does not limit the present invention. The same portions in the drawings are denoted by the same reference numerals, and detailed description thereof is omitted suitably, and different portions will be described. Note that the drawings are schematic or conceptual, and the relationship between the thickness and the width of each portion, the ratio of the sizes between the portions, and the like are not necessarily the same as actual ones. In addition, even in the case of representing the same portions, respective drawings may represent the dimensions and ratios of the same portions differently from one another.

### First Embodiment

### <Thermal Conductivity Gas Sensor System>

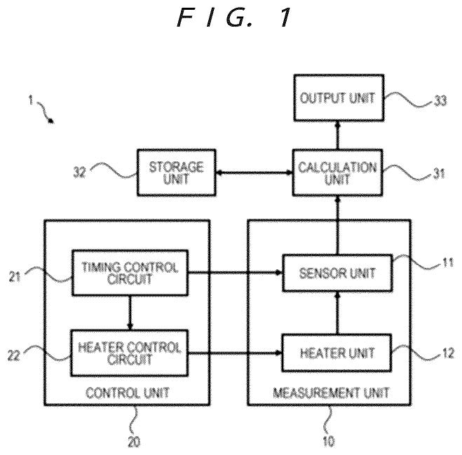

FIG. 1 is a schematic view of a thermal conductivity gas sensor system 1 according to the first embodiment.

The thermal conductivity gas sensor system 1 according to the first embodiment includes a measurement unit 10, a control unit 20, and a calculation unit 31.

The measurement unit 10 includes a sensor unit 11 and a heater unit 12.

The sensor unit 11 includes a resistor. The sensor unit 11 can measure the resistance value (resistance value) of the resistor. The acquired resistance value is sent to the calculation unit 31.

The heater unit 12 can heat the sensor unit 11. The heater unit 12 includes, for example, a micro heater.

The control unit 20 includes a timing control circuit 21 and a heater control circuit 22.

The timing control circuit 21 issues a control signal for controlling heating and/or non-heating of the heater unit 12 to the heater control circuit 22. In addition, the timing control circuit 21 issues a control signal for controlling the timing for measuring an output signal acquired from the sensor unit 11 to the sensor unit 11. In addition to the control of these timings, the timing control circuit 21 performs control necessary for measurement, for example, synchronization of time at which measurement is performed.

The heater control circuit 22 controls the heating and the non-heating of the heater unit 12. The method of controlling the heater control circuit 22 is not limited to one method. For example, the control may be performed by turning on and off a current or voltage of the heater unit 12, or the value of the current or voltage flowing to the heater unit 12 may be subjected to PID control.

The calculation unit 31 acquires a first output signal from the sensor unit 11 when the heater unit 12 is not heated, and acquires a second output signal from the sensor unit 11 when the heater unit 12 is heated. The calculation unit 31 performs calculation to correct a measurement result using the acquired first output signal and second output signal.

Here, it is preferable to acquire each output signal after the output signal to be acquired from the sensor unit 11 is “stabilized”. Here, “stabilized” means a state in which the fluctuation range of the output of the sensor unit 11 falls within a predetermined range, for example, within ±1% of the average value of the output signals of the sensor unit 11, in a certain period.

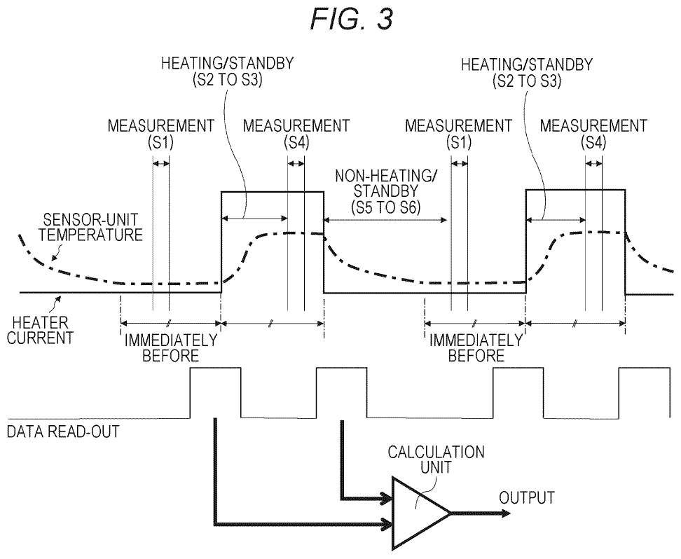

Here, the timing of acquiring the first output signal is preferably immediately before the heater unit 12 heats the sensor unit 11. This is because, as the timing at which the first output signal is acquired is before and closer to the timing at which the heating is started, the influence due to a change in the environment in which the thermal conductivity gas sensor system 1 is placed is smaller, the influence from the change in the environment at the timing at which the first output signal and the second output signal are measured does not need to be considered much, and a measurement result closer to a true value can be acquired. In addition, by setting the timing at which the first output signal is acquired to immediately before, the time interval from the end of heating by the heater unit 12 to the timing at which the first output signal is acquired can be made long, so that measurement can be performed in a state where the temperature change is smaller, and thus improvement in accuracy can be expected. Here, “immediately before” is determined according to a measurement system and measurement conditions, and means any of temporal points before the heater unit 12 is heated and at which the condition is satisfied that the fluctuation range of the output of the sensor unit 11 falls within a predetermined range, for example, within ±1% of the average value of the output signals of the sensor unit 11, in a certain period. Here, the certain period means, for example, the same time length as the time length required for measurement at the time of heating. In addition, for example, any of the temporal points in the range indicated as “immediately before” in FIG. 3 is exemplified.

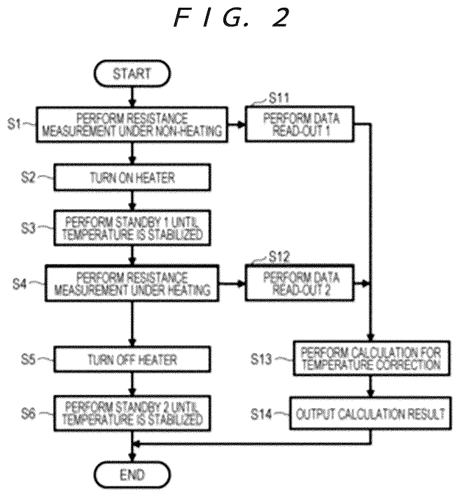

The thermal conductivity gas sensor system 1 may include a storage unit 32. For example, the storage unit 32 may temporarily store the first signal output and the second signal output, and may output the first signal output and the second signal output to the calculation unit 31 as necessary, or store a calculation result.

[Sensing Method According to First Embodiment]Hereinafter, a measurement method using the thermal conductivity gas sensor system according to the present embodiment will be described with reference to FIGS. 2 and 3.

### (S1, S11)

After the temperature of the sensor unit 11 is stabilized under non-heating, the timing control circuit 21 issues a control signal for instructing to measure the resistance value of the sensor unit 11 to the sensor unit 11. The acquired value (first output signal) is output to the calculation unit 31.

### (S2 to S3)

Next, the heater control circuit 22 issues a control signal for instructing to heat to the heater unit 12. As the heater unit 12 is heated, the temperature of the sensor unit 11 also increases. Standby is performed until the temperature of the sensor unit 12 is stabilized.

### (S4, S12)

After the temperature of the sensor unit 11 is stabilized under a heating state, the timing control circuit 21 issues a control signal for instructing to measure the resistance value of the sensor unit 11 to the sensor unit 11. The acquired value (second output signal) is output to the calculation unit 31.

### (S5, S6)

The heater control circuit 22 issues a control signal for instructing not to heat to the heater unit 12. As the temperature of the heater unit 12 decreases, the temperature of the sensor unit 11 also decreases. Standby is performed until the temperature of the sensor unit 11 is stabilized.

### (S13, S14))

Calculation for temperature correction is performed using the values (first output signal and second output signal) acquired in S11 and S12. The acquired calculation result is output to the storage unit 32 and an output unit 33 such as a monitor.

A series of steps S1 to S14 as a cycle of one unit is repeatedly performed, whereby sensing over time becomes possible.

### Modification of First Embodiment

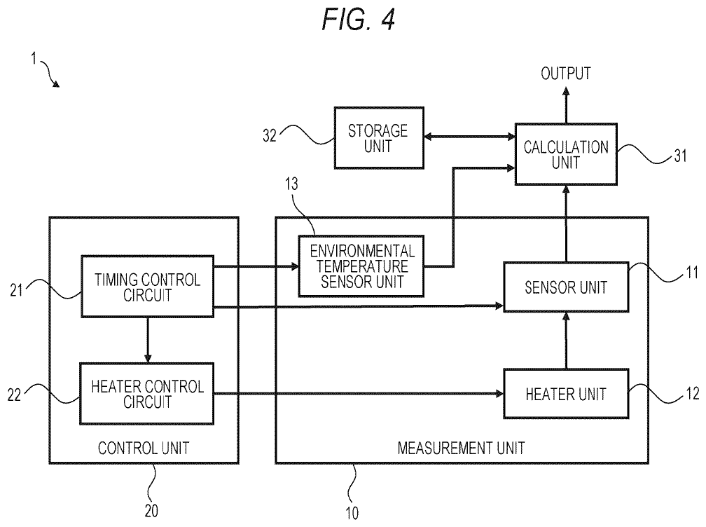

FIG. 4 is a schematic view of a thermal conductivity gas sensor system 1 according to a modification of the first embodiment. The present modification includes an environmental temperature sensor unit 13 in addition to the first embodiment. By further including the environmental temperature sensor unit 13, more accurate sensing can be performed.

At this time, the temperature dependency of the sensor unit 11 is preferably lower than the temperature dependency of the environmental temperature sensor unit 13. Here, the temperature dependency refers to a ratio of sensor output voltage fluctuation to temperature change.

Since the temperature dependency of the sensor unit 11 is lower than the temperature dependency of the environmental temperature sensor unit 13, the response fluctuation of the sensor unit 11 with respect to the temperature change becomes small, so that the sensor output is stabilized. In addition, the change (temperature dependency) of the environmental temperature sensor unit 13 with respect to the environmental temperature change becomes high, so that also the improvement of the environmental temperature monitoring accuracy can be achieved.

The environmental temperature sensor unit 13 and the sensor unit 11 are preferably included in the same package. By including the environmental temperature sensor unit 13 and the sensor unit 11 in the same package, the measurement environments for both can be the same or can have approximate conditions, the common mode noise can be removed, and the influence of the external environment can be further reduced. Therefore, more accurate sensing can be performed.

[Sensing Method according to Modification of First Embodiment]

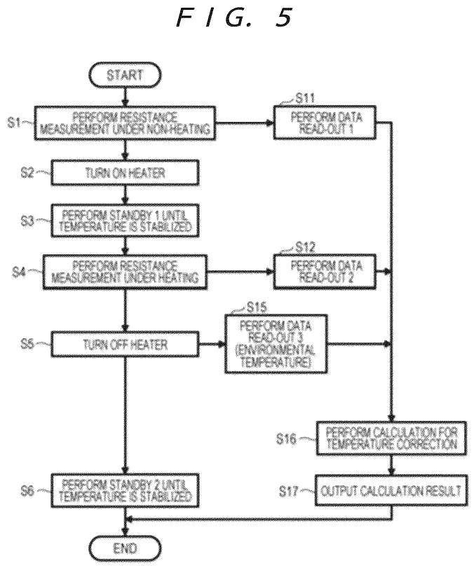

Since S1 to S6 and S11 to S12 are the same as those of the first embodiment, description thereof is omitted, and points different from the first embodiment will be described with reference to FIG. 5.

### (S15)

After the heater control circuit 22 issues a control signal for instructing not to heat to the heater unit 12, the timing control circuit 21 issues a control signal for instructing to measure the resistance value of the environmental temperature sensor unit 13 to the environmental temperature sensor unit 13. The acquired value (third output signal) is output to the calculation unit 31.

### (S16, S17)

Calculation for temperature correction is performed using the values (first output signal, second output signal, and third output signal) acquired in S11, S12, and S15. The acquired calculation result is output to the storage unit 32 and the output unit 33 such as a monitor.

### EXAMPLES

Examples of the embodiments will be described below.

### Example 1

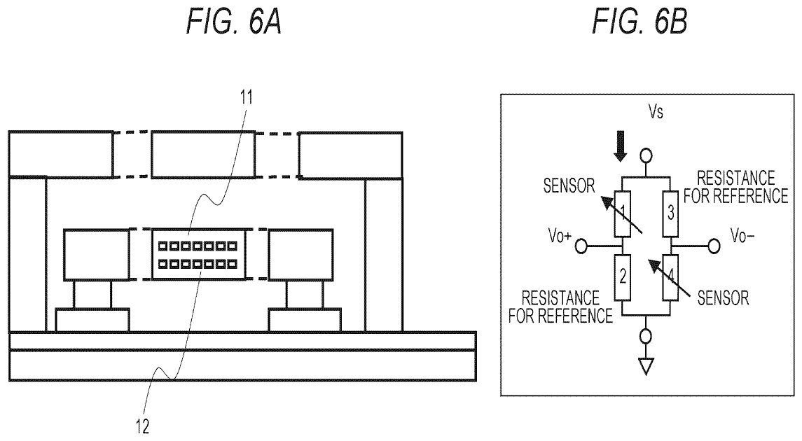

Example 1 according to the first embodiment will be described below. Here, the measurement was performed using the thermal conductivity gas sensor system illustrated in FIG. 6A.

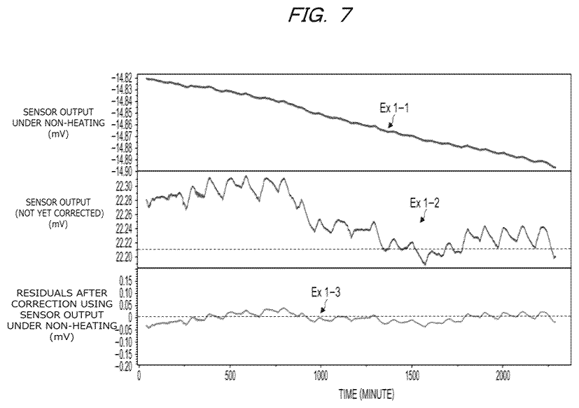

First, the power of the heater unit 12 was turned off, and after the temperature of the sensor unit 11 was stabilized under non-heating, the timing control circuit 21 issued the control signal for instructing to measure the resistance value of the sensor unit 11 to the sensor unit 11, and the resistance value at that time was measured. The acquired value (first output signal) was output to the calculation unit 31. The first output signals acquired at that time are plotted as Ex1-1 in FIG. 7.

Next, the heater control circuit 22 issued the control signal for instructing to heat to the heater unit 12. As the heater unit 12 was heated, the temperature of the sensor unit 11 also increased. Then, standby was performed until the temperature of the sensor unit 11 was stabilized. After the temperature of the sensor unit 11 was stabilized, the timing control circuit 21 issued the control signal for instructing to measure the resistance value of the sensor unit 11 to the sensor unit 11, and the resistance value at that time was measured. The acquired value (second output signal) was output to the calculation unit 31. The second signal outputs acquired at that time are plotted as Ex1-2 in FIG. 7.

Calculation for temperature correction was performed using the values (first output signal and second output signal) acquired as mentioned above. Specifically, a correction value calculated from Ex1-1 based on the pre-measured calibration curve was subtracted from Ex1-2. The residuals at that time are plotted as Ex1-3.

The fluctuation range of the residual Ex1-3 with respect to the sensor output was within the range of ±0.3%. From this, it was indicated that highly accurate measurement was possible despite having only one sensor for measuring the temperature in Example 1.

### Example 2

Example 2 corresponding to a modification of the first embodiment will be described. Here, measurement was performed by adding an environmental temperature sensor unit 13 (not illustrated in FIGS. 6A and 6B) to the thermal conductivity gas sensor system of Example 1 (FIG. 6A). A bridge circuit illustrated in FIG. 6B is constituted with the sensor unit 11 and the environmental temperature sensor unit 13.

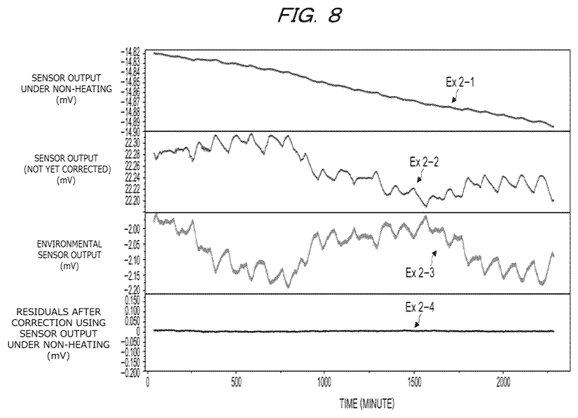

Until the first output signal and the second output signal were acquired, the same steps in Example 1 were carried out. The first output signals Ex2-1 and second output signals Ex2-2 acquired at that time are plotted in FIG. 8.

After the second output signal was acquired, the heater control circuit 22 issued the control signal for instructing not to heat to the heater unit 12. Then, the timing control circuit 21 issued the control signal for instructing to measure the resistance value of the environmental temperature sensor unit 13 to the environmental temperature sensor unit 13, and the resistance value at that time was measured. The acquired value (third output signal) was output to the calculation unit 31. The third output signals acquired at that time are plotted as Ex2-3 in FIG. 8.

Calculation for temperature correction is performed using the values (first output signal, second output signal, and third output signal) acquired in S11, S12, and S15. Specifically, correction values calculated from the first and third output signals based on the pre-measured calibration curve were subtracted from the second output signal. The residuals at that time are plotted as Ex2-4.

The fluctuation range of the residual Ex2-4 with respect to the sensor output was within the range of ±0.01%. From this, it was indicated that the measurement in Example 2 could be performed with higher accuracy than that in Example 1.

### Comparative Example

A comparative example will be described below. In the present comparative example, a thermal conductivity gas sensor system 1 similar to that of Example 2 was used.

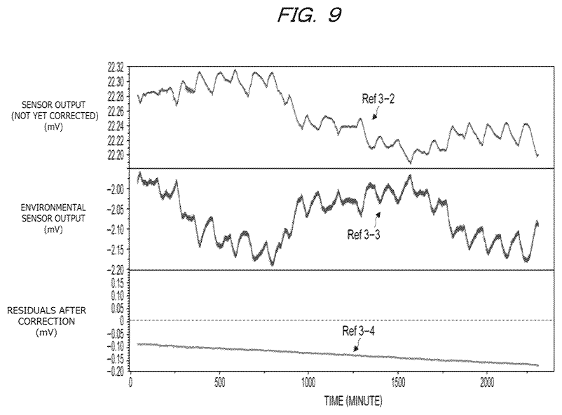

Without performing S1 and S11, the heater control circuit 22 issued the control signal for instructing to heat to the heater unit 12. As the heater unit 12 was heated, the temperature of the sensor unit 11 also increased. Then, standby was performed until the temperature of the sensor unit 11 was stabilized. After the temperature of the sensor unit 11 was stabilized, the timing control circuit 21 issued the control signal for instructing to measure the resistance value of the sensor unit 11 to the sensor unit 11, and the resistance value at that time was measured. The acquired value (second output signal) was output to the calculation unit 31. The second signal outputs acquired at that time are plotted as Ref3-2 in FIG. 9.

Next, after the second output signal was acquired, the heater control circuit 22 issued the control signal for instructing not to heat to the heater unit 12. Then, the timing control circuit 21 issued the control signal for instructing to measure the resistance value of the environmental temperature sensor unit 13 to the environmental temperature sensor unit 13, and the resistance value at that time was measured. The acquired value (third output signal) was output to the calculation unit 31. The third output signals acquired at that time are plotted as Ref3-3 in FIG. 9.

Calculation for temperature correction was performed using the values (second output signal and third output signal) acquired as mentioned above. Specifically, Ref3-3 was subtracted from Ref3-2. The residuals at that time are plotted as Ref3-4.

The residuals Ref3-4 were not stabilized over time, and the fluctuation range with respect to the sensor output did not fall within the range of ±18. From this, it was indicated that the accuracy of the thermal conductivity gas sensor system in the comparative example was lower than that in Example 1 and Example 2.

Furthermore, an embodiment may include the following configurations.

### [Configuration 1]

A thermal conductivity gas sensor system including:
a measurement unit that includes
   a sensor unit capable of measuring a resistance value and
   a heater unit capable of heating the sensor unit;
a control unit that includes
   a heater control circuit capable of controlling heating and non-heating of the heater unit and
   a timing control circuit capable of controlling timing of a control signal for heating and non-heating of the heater unit and timing of an output signal from the sensor unit; and
a calculation unit that is configured to acquire a first output signal from the sensor unit when the heater unit is not heated and correct a second output signal from the sensor unit when the heater unit is heated using the first output signal.

### [Configuration 2]

The thermal conductivity gas sensor system according to Configuration 1, in which a timing of acquiring the first signal is immediately before the heater unit heats the sensor unit.

### [Configuration 3]

The thermal conductivity gas sensor system according to Configuration 1 or 2, in which the thermal conductivity gas sensor system repeatedly performs a cycle as one unit including:
a step of acquiring a first output signal of the sensor unit with the calculation unit immediately before heating the heater unit;
a step of heating the heater unit with the heater control circuit after the acquisition is finished, and performing a first standby with the timing control circuit until a temperature of the sensor unit is stabilized;
a step of acquiring a second output signal of the sensor unit with the calculation unit after the first standby is finished;
a step of terminating the heating of the heater unit with the heater control circuit after the acquisition of the second output signal is finished, and performing a second standby with the timing control circuit until the temperature is stabilized; and
the thermal conductivity gas sensor system performs, after the second standby is performed in the previous cycle, acquisition of a first signal output of the first sensor unit in the next cycle.

### [Configuration 4]

The thermal conductivity gas sensor system according to any one of Configurations 1 to 3, further including an environmental temperature sensor.

### [Configuration 5]

The thermal conductivity gas sensor system according to Configuration 4 in which the environmental temperature sensor and the sensor unit are included in the same package.

### [Configuration 6]

A sensing method for a thermal conductivity gas sensor system including:
a measurement unit that includes
   a sensor unit capable of measuring a resistance value and
   a heater unit capable of heating the sensor unit;
a control unit that includes
   a heater control circuit capable of controlling heating and non-heating of the heater unit and
   a timing control circuit capable of controlling timing of a control signal for heating and non-heating of the heater unit and timing of an output signal from the sensor unit; and
a calculation unit that is configured to acquire a first output signal from the sensor unit when the heater unit is not heated and correct a second output signal from the sensor unit when the heater unit is heated using the first output signal, the sensing method allowing the thermal conductivity gas sensor system to repeatedly perform a cycle as one unit including:
a step of acquiring a first output signal of the sensor unit with the calculation unit immediately before heating the heater unit;
a step of heating the heater unit with the heater control circuit after the acquisition is finished, and performing a first standby with the timing control circuit until a temperature of the sensor unit is stabilized;
a step of acquiring a second output signal of the sensor unit with the calculation unit after the first standby is finished; and
a step of terminating the heating of the heater unit with the heater control circuit after the acquisition of the second output signal is finished, and performing a second standby with the timing control circuit until the temperature is stabilized.

With an acidic gas absorbent, an acidic gas removal method, and an acidic gas removal system of at least one embodiment described above, it is possible to implement an acidic gas absorption system with less waste and reduced energy required for regeneration.

Although some embodiments of the present invention have been described, these embodiments have been presented as examples, and are not intended to limit the scope of the invention. These novel embodiments can be implemented in various other forms, and various combinations, omissions, substitutions, and changes can be made without departing from the gist of the invention. These embodiments and modifications thereof are included in the scope and gist of the invention, and are included in the invention described in the scope of the claims and the equivalent scope thereof.

What is claimed is:

1. A thermal conductivity gas sensor system comprising:
a measurement unit that includes
a sensor unit capable of measuring a resistance value and
a heater unit capable of heating the sensor unit;
a control unit that includes
a heater control circuit capable of controlling heating and non-heating of the heater unit and
a timing control circuit capable of controlling timing of a control signal for heating and non-heating of the heater unit and timing of an output signal from the sensor unit; and
a calculation unit that is configured to acquire a first output signal from the sensor unit when the heater unit is not heated and correct a second output signal from the sensor unit when the heater unit is heated using the first output signal.

2. The thermal conductivity gas sensor system according to claim 1, wherein a timing of acquiring the first signal is immediately before the heater unit heats the sensor unit.

3. The thermal conductivity gas sensor system according to claim 1, wherein the thermal conductivity gas sensor system repeatedly performs a cycle as one unit including:
a step of acquiring a first output signal of the sensor unit with the calculation unit immediately before heating the heater unit;
a step of heating the heater unit with the heater control circuit after the acquisition is finished, and performing a first standby with the timing control circuit until a temperature of the sensor unit is stabilized;
a step of acquiring a second output signal of the sensor unit with the calculation unit after the first standby is finished;
a step of terminating the heating of the heater with the heater control circuit after the acquisition of the second output signal is finished, and performing a second standby with the timing control circuit until the temperature is stabilized; and
the thermal conductivity gas sensor system performs, after the second standby is performed in the previous cycle, acquisition of a first signal output of the first sensor unit is performed in the next cycle.

4. The thermal conductivity gas sensor system according to claim 1, further comprising an environmental temperature sensor.

5. The thermal conductivity gas sensor system according to claim 4 wherein the environmental temperature sensor and the sensor unit are included in a same package.

6. A sensing method for a thermal conductivity gas sensor system including:
a measurement unit that includes
a sensor unit capable of measuring a resistance value and
a heater unit capable of heating the sensor unit;
a control unit that includes
a heater control circuit capable of controlling heating and non-heating of the heater unit and
a timing control circuit capable of controlling timing of a control signal for heating and non-heating of the heater unit and timing of an output signal from the sensor unit; and
a calculation unit that is configured to acquire a first output signal from the sensor unit when the heater unit is not heated and correct a second output signal from the sensor unit when the heater unit is heated using the first output signal, the sensing method allowing the thermal conductivity gas sensor system to repeatedly perform a cycle as one unit comprising:
a step of acquiring a first output signal of the sensor unit with the calculation unit immediately before heating the heater unit;
a step of heating the heater unit with the heater control circuit after the acquisition is finished, and performing a first standby with the timing control circuit until a temperature of the sensor unit is stabilized;
a step of acquiring a second output signal of the sensor unit with the calculation unit after the first standby is finished; and
a step of terminating the heating of the heater unit with the heater control circuit after the acquisition of the second output signal is finished, and performing a second standby with the timing control circuit until the temperature is stabilized.
